(19) United States
(12) Patent Application Publication
(10) Pub. No.: US 2019/0015326 A1
(43) Pub. Date: Jan. 17, 2019

(54) Novel Bioavailability Enhancement Composition

(71) Applicant: ExxPharma Therapeutics LLC, Morris Plains, NJ (US)

(72) Inventors: Isaac Ghebre-Sellassie, Morris Plains, NJ (US); Hibreniguss Terefe, Piscataway, NJ (US)

(21) Appl. No.: 16/032,256

(22) Filed: Jul. 11, 2018

Related U.S. Application Data
(60) Provisional application No. 62/531,826, filed on Jul. 12, 2017.

(51) Int. Cl. A61K 9/10; A61K 47/38; A61K 47/32; A61K 47/10; A61K 47/02; A61K 47/44; A61K 47/26; A61K 47/34

(52) U.S. Cl. CPC A61K 9/10; A61K 47/38; A61K 47/32; A61K 47/34; A61K 47/02; A61K 47/44; A61K 47/26; A61K 47/10

(57) ABSTRACT

The invention provides a novel bioavailability enhancement oral solid dosage form composition and process that enhances the solubility of all poorly soluble drug substances irrespective of their physicochemical properties. The composition comprising a drug substance, at least one solid hydrophilic polymer, at least one high boiling liquid, at least one emulsifier, at least one anti-nucleating agent, and at least one anti-adherent, and the manufacturing process utilizes a high intensity mixer with counter-rotating or co-rotating screw elements.

### FIELD OF INVENTION

The present invention relates to oral solid dosage form composition that enhances the bioavailability of poorly water-soluble drug substances. More particularly, it pertains to a novel composition that enhances the bioavailability of poorly soluble drug substances irrespective of their physicochemical properties.

### BACKGROUND OF THE INVENTION

One enormous challenge in drug development is the increasing number of poorly soluble drug candidates emerging from discovery pipelines. Approximately 90% of the drug candidates in the pipelines are poorly soluble, a figure that is significantly higher than the less than 40% of poorly soluble, marketed drugs. Insufficient bioavailability due to poor formulation can lead to poor and variable bioavailability, suboptimal clinical performance, delays in development or even attrition of promising drug substances from the development pipeline. As new chemical entities in the pipeline are increasingly becoming more and more poorly water soluble and lipophilic, development of viable dosage forms for such molecules continues to pose many challenges. Various solubilization techniques have been employed to address the challenges, including particle size reduction, lipid-based formulations and amorphous solid dispersions, the latter being the most popular.

Particle Size Reduction:

The bioavailability of low solubility drugs is often intrinsically related to particle size. Particle size reduction results in increased surface area, thereby improving the dissolution rate of the drug substance.

U.S. Pat. No. 5,704,556 discloses a media milling process for producing colloidal particles using ceramic beads of less than 100 microns in diameter in which the diameter of the ceramic milling media beads is no more than about one hundred times the average particle size of the feedstock particles.

U.S. Pat. No. 8,092,785 describes a pharmaceutical formulation preparation by a process, which comprises co-milling of a suspension of the drug substance and a pharmaceutically acceptable polymer in a liquid propellant.

EP A 0330532 describes the co-micronization of fenofibrate with a surfactant, preferably sodium lauryl sulfate, which composition is said to have improved bioavailability.

Methods of particle size reduction rely upon mechanical stress. However, the mechanical forces inherent to comminution, such as milling, micronization and grinding, often impart significant amount of physical stress on the drug substance potentially inducing degradation. The thermal stress, which may occur during size reduction, is also a concern when processing thermo-sensitive or unstable drug substances compounds.

Lipid-Based Formulations:

Lipid-based formulations have also been used to improve oral bioavailability of poorly water-soluble compounds. The approach involves, dissolution of the lipophilic drug in oils, followed by emulsification of the oil in an aqueous phase. The composition is expected to maintain the drug in the solubilized state in the gastrointestinal tract until absorption occurs. Thus, lipid formulations, which comprise a variety of excipients, are designed to generate and maintain a supersaturated drug concentration in vivo for improved oral absorption. One of the most critical criteria in lipid-based formulations is the drug solubilization capacity of the excipients. The excipients must be able to fully solubilize the entire drug dose, preferably in a volume for a single dosage. In addition to the solubilization capacity, excipients in the lipid-based formulations must maintain the drug in the solubilized state. The metastable supersaturated drug concentration needs to be maintained for a long enough time-period for absorption to occur before drug precipitation. This requires the incorporation of significant quantities of surfactants and co-solvents to achieve the targeted dose level and form stable microemulsions, which in turn leads to low drug loadings. In addition, large quantities of surfactants and co-solvents tend to pose safety concerns. These factors limit the number and types of excipients that have high solubilization capacity for lipophilic drugs as well as acceptable toxicity profiles.

Sometimes, precipitation inhibitors are incorporated in the formulation to help inhibit drug precipitation in the gastrointestinal tract, which further reduces the drug loading and increases the unit dosage size to a level that could pose a swallowing challenge to patients.

U.S. Pat. No. 9,511,078 describes a pharmaceutical formulation of liquid oral dosage form containing a low aqueous solubility drug, a solubilizer, an emulsifier, and optionally at least one additional pharmaceutically acceptable excipient, formulated in a capsule. The drug is solubilized within the other components of the pharmaceutical formulation, which forms a nano-emulsion upon exposure to an aqueous environment, such as the gastrointestinal tract, where a mean particle size (D50) of a droplet of the emulsion typically ranges from about 50 nm to about 800 nm. A typical example of a liquid-filled capsule formulation given in the patent contains only 6% Fenofibrate in addition to 80% glycerol caprylate, 8.9% polyoxy-castor oil, 5% hydroxy propyl cellulose and 0.05% d-α-tocopherol.

U.S. Pat. No. 9,089,534 B2 describes a semi-solid composition of Isotretinoin consisting of at least two lipid excipients, one of them being hydrophilic (an HLB value higher or equal to 10), the other an oily vehicle. A typical example in the patent consists of 10-20 mg isotretinoin, 40-270 mg soybean oil, 84-228 mg Gelucire, 50/13 in 400 mg unit dose.

International Patent Publication WO 2002007712 A2 discloses an oral liquid self-emulsifying drug delivery system for extremely water-insoluble, lipophilic compounds. The self-emulsifying drug delivery system comprises a mixture of an extremely water-insoluble, lipophilic active agent; polyvinyl pyrrolidone; a fatty acid; and a surfactant wherein, the weight ratio of said fatty acid to said polyvinyl pyrrolidone is about 2:1 to about 1:3, and the weight ratio of said surfactant to said polyvinyl pyrrolidone is about 10:1 to about 1:1.

Most commercially available lipid-based formulations are liquids or semi-solids that are filled in capsules, compositions that are susceptible to physical and/or chemical instability. In addition, lipid formulations are not suitable for hydrophilic drug substances.

Amorphous Solid Dispersions:

Amorphous solid dispersions refer to dispersions of one or more active ingredients in inert carriers or matrices at solid state that are employed to enhance the solubility and hence bioavailability of poorly soluble drug substances. They improve bioavailability by increasing the rate and extent of dissolution of the drug substances. As a result, amorphous solid dispersions have become of tremendous interest to the industry.

U.S. Pat. No. 7,713,548 relates to amorphous solid dispersions of 7-chloro-N, N,5-trimethyl-4-oxo-3-phenyl-3,5-dihydro-4H-pyridazino[4,5-b] indole-1-acetamide, a pharmacological agent possessing a high affinity for the peripheral-type benzodiazepine receptors, and processes for the preparation of these amorphous solid dispersions.

U.S. Pat. No. 7,364,752 B1 relates to a pharmaceutical composition comprising a solid dispersion of an HIV protease inhibitor in a water-soluble carrier, such as PEG, having enhanced bioavailability and improved dissolution properties. The solid dispersion may optionally be encapsulated in hard gelatin capsules, compressed into a tablet, or may be granulated with a pharmaceutically acceptable granulating agent. Also disclosed are methods of making said solid dispersion.

U.S. Pat. No. 5,456,923 describes a process for producing a solid dispersion, which employs a twin-screw extruder. A solid dispersion is produced without heating a drug and a polymer up to or beyond their melting points and without the use of organic solvents to dissolve the components.

EP 1027885 A2 relates to a composition comprising a basic drug, a drug which forms a zwitterion, or a salt of either entity, admixed with a polymer selected from hydroxypropylmethylcellulose acetate succinate, cellulose acetate trimellitate, cellulose acetate phthalate, hydroxypropylcellulose acetate phthalate, hydroxypropylmethyl-cellulose acetate phthalate, and methylcellulose acetate phthalate, all of which are entero-soluble polymers.

In spite of the popularity of amorphous solid dispersions in pharmaceutical product development, they do not overcome effectively the poor bioavailabilities of many molecular entities, particularly poorly soluble weak bases and acids (pkₐ); and highly lipophilic drug substances (log P).

The object of the present invention is to describe a novel solid oral dosage form composition and process that enhances the bioavailability of poorly soluble drug substances and overcomes the short comings of particle size reduction, lipid formulations and amorphous solid dispersions.

### SUMMARY OF THE INVENTION

The invention relates to a novel solubility enhancement process and composition comprising a poorly soluble drug substance, at least one solid hydrophilic polymer, at least one high boiling liquid, at least one emulsifier, at least one anti-nucleating agent and at least one anti-adherent that are mixed in specific ratios in a high intensity mixer. The physicochemical properties of the poorly soluble drug substance determine the relative amounts of the components in the composition and manufacturing conditions, both of which are critical to achieving solubility/bioavailability enhancement.

In the invention, it is prerequisite that the ratio of one component to another in the composition falls within a specific range for the composition to be effective. Hence the relative amounts are expressed as a ratio by weight between two components to establish boundaries as follows:
(a) the weight ratio of the amount of the emulsifier relative to the amounts of solid hydrophilic polymer is 1:50 to 50:1;
(b) the weight ratio of the amount of the emulsifier relative to the amount of the high boiling liquid is 1:20 to 20:1;
(c) the weight ratio of the amount of the emulsifier relative to the amounts of the anti-nucleating agent is 1:50 to 50:1;
(d) the weight ratio of the amount of the high boiling liquid relative to the amount of the anti-nucleating agent is 1:20 to 20:1;
(e) the weight ratio of the amount of the high boiling liquid relative to the amount of the solid hydrophilic polymer is 1:50 to 50:1;
(f) the weight ratio of the amount of anti-nucleating agent relative to the amount of solid hydrophilic polymer is 1:1 to 1:200;
(g) the amount of the anti-adherent relative to the amount of the total composition by weight is between 0.1 to 10%; and
(h) the amount of drug substance relative to the amount of the total composition by weight is between 1% and 70%.
It is also essential that all the components in their respective ratios are present in the composition to obtain the desired results, although in special cases one or more components may be omitted or added.

### DETAILED DESCRIPTION OF THE INVENTION

Poor solubility of drug substances leads to low dissolution rate and in turn to low bioavailability following oral administration. 90% of drug substances in the discovery pipeline are poorly soluble. The common molecular characteristics of drug substances that impact solubility are strong molecular forces and hydrophobicity. Very strong molecular forces lead to very strong crystal lattice that is exhibited by high melting point and low solubility. A T, of 200° C. has been identified as the cut-off value for crystal lattice to have strong influence on the solubility. High hydrophobicity is exhibited by high log P (Octanol/water partition coefficient) and low aqueous solubility. Hydrophobic compounds have a limited capacity to interact with the water phase, in accordance with similia similibus solvuntur (like dissolves like), and these compounds are solubility-limited due to poor hydration. Log P values of 2-3 have been recommended as the cut-off point for hydration which imposes significant limitation on solubility; the higher the value, the poorer the hydration. For ionizable drug substances, the log D (partition coefficient at the pH of interest) should be greater than the cut-off value of log P.

Techniques such as particle size reduction (micronization, nano-suspensions, nano-crystallization), and modification of crystal habits (polymorphs, pseudo-polymorphs amorphous solid dispersions) in principle solve solubility limitations due to low surface area considerations; however, they do not effectively solve solubility problems if the molecules have very strong crystal lattice and/or are highly hydrophobic.

Amorphous solid dispersions are employed to convert poorly soluble crystalline drug substances into an amorphous form. The common techniques used to prepare amorphous solid dispersions are melt extrusion and spray drying. The use of melt extrusion is limited by melting point of the drug substance, with a reported cut-off point being 200° C. Spray drying is limited by the drug solubilization capacity of a volatile solvent, which has a cutoff point of 10 mg/ml. Moreover, amorphous solid dispersions do not offer a solution to hydrophobic substances, since precipitation of the drug substance occurs as soon it comes in contact with an aqueous medium, which, in turn, negatively impacts bioavailability and stability.

Hydrophobic drug substances are preferably formulated using lipid-based vehicles comprising lipids, surfactants and/or cosolvents, although these approaches are limited by drug loading, instability and safety concerns.

Unfortunately, compounds with high melting points and high log P values display both solid-state- and solvation-limited solubility and typically aggregate or precipitate out of solution. Such compounds are very difficult to manipulate, even by altering the formulation, and are likely to produce concentrations in vivo that are too low to have therapeutic effects. Hence, there is a need for the development of compositions and processes that enhance solubility of poorly soluble drug substances irrespective their physicochemical properties.

It is the object of the invention, therefore, to describe a process and composition that enhance the bioavailability of all poorly drug substances irrespective of their physicochemical properties including melting point (M.P.), dissociation constant (pkₐ) and octanol-water partition coefficient (log P).

In one embodiment, the invention relates to a composition comprising a drug substance, a solid hydrophilic polymer, a high boiling liquid, an emulsifier, an anti-nucleating agent, and an anti-adherent having the following ratios:
(a) the weight ratio of the amount of the emulsifier relative to the amounts of solid hydrophilic polymer is 1:50 to 50:1 preferably 1:20 to 20:1;
(b) the weight ratio of the amount of the emulsifier relative to the amount of the high boiling liquid is 1:20 to 20:1 preferably 1:10 to 10:1;
(c) the weight ratio of the amount of the emulsifier relative to the amounts of the anti-nucleating agent is 1:50 to 50:1 preferably 1:20 to 20:1;
(d) the weight ratio of the amount of the high boiling liquid relative to the amount of the anti-nucleating agent is 1:20 to 20:1 preferably 1:10 to 10:1;
(e) the weight ratio of the amount of the high boiling liquid relative to the amount of the solid hydrophilic polymer is 1:50 to 50:1 preferably 1:20 to 20:1;
(f) the weight ratio of the amount of anti-nucleating agent relative to the amount of solid hydrophilic polymer is 1:200 to 1:1 preferably 1:100 to 1:1;
(g) the amount of the anti-adherent relative to the amount of the total composition by weight is between 0.1 to 20% preferably 0.5 to 10%; and
(h) the amount of drug substance relative to the amount of the total composition by weight is between 1% and 70% preferably 5 to 50%.

The composition is mixed using a high intensity mixer under controlled pressures and temperatures.

In another embodiment, the invention relates to a composition that contains poorly drug substances, which are classified as BCS II (poorly soluble, highly permeable), BCS III (highly soluble, poorly permeable) and BCS IV (poorly soluble, poorly permeable) according to Biopharmaceutical Classification System (BCS).

In yet another embodiment, the drug substances include, but not limited to, acetazolamide, azathioprine, amphoterecin B, aprepitant, carbamazepine, carvedilol, cilostazol, cyclosporine, danzol, etavirine, famotidine, furosemide, hydrochlorthiazide, hydroxyzine, ibuprofen, indinavir, itraconazole, ketoconazole, ketoprofen, lopinavir, meloxicam, nelfinavir, piroxicam, ritonavir, saquinavir, or combinations thereof.

In another embodiment, the invention relates to a composition that contains solid hydrophilic polymers which include, but not limited to, cellulosic derivatives, such as hydroxypropylmethyl cellulose; hydroxypropylcellulose and hydroxypropylmethyl cellulose acetyl succinate, vinyl derivatives such as polyvinyl pyrrolidone; polyvinyl pyrrolidone—vinyl acetate co-polymer; polyethylene glycol—polyvinyl acetate and polyvinyl caprolactam-based graft copolymer, or mixtures thereof.

In yet another embodiment, the invention relates to a composition that contains high boiling liquids which include, but not limited to, polyethylene glycol, propylene glycol, diethylene glycol monoethyl ether or mixtures thereof.

In yet another embodiment, the invention relates to a composition that contains emulsifiers which include, but not limited to, propylene glycol caprylate, propylene glycol dicaprylocaprate, linolyl polyoxyglycerides, caprylcaproyl polyoxyglycerides, propylene glycol dicaprylocaprate, propylene glycol laurate, polyglyceride oleate, sorbitan monolaurate, sorbitan monooleate, or mixtures thereof.

In yet another embodiment, the invention relates to a composition that contains anti-nucleating agents which include, but not limited to, low molecular weight polymers such as hydroxypropyl methylcellulose, hydroxypropyl cellulose, α-Hydro-ω-hydroxypoly(oxyethylene)-poly(oxypropylene).poly(oxyethylene) block copolymer, polyvinyl pyrrolidone, polyvinyl pyrrolidone vinyl acetate; surfactants such as polyethylene glycol-20 sorbitan monolaurate, polyethylene glycol-20 monostearate, polyethylene glycol-20 sorbitan monooleate or mixtures thereof.

In yet another embodiment, the invention relates to a composition that contains anti-adherents which include, but not limited to, silicon dioxide, talc, kaolin, titanium dioxide, aluminum magnesium silicate, or mixtures thereof. Optionally, other pharmaceutical aids such as antioxidants, buffering agents, sweetening agents, etc. are added to the formulations, if needed, to enhance stability or improve taste as is well known in the art.

In yet another embodiment, the invention relates to a process of preparing a composition for enhancing bioavailability of poorly soluble drug substances. The process comprises using an extrusion process to mix the drug substance, the solid hydrophilic polymer, the high boiling liquid, the emulsifier, the anti-nucleating agent, and the anti-adherent. Extrusion may use a twin-screw high intensity mixer that utilizes co-rotating or counter-rotating screw elements.

The invention is further illustrated, in part, but not limited to, by the following examples which demonstrate the effectiveness of the composition in enhancing the solubility/dissolution rate of poorly soluble drug substances:

### Example 1

A composition containing a mixture of a poorly soluble drug substance, a solid hydrophilic polymer, a low boiling liquid, an emulsifier, anti-nucleating agent and an anti-adherent was blended/granulated. The blend/granulation was then fed into a twin-screw extruder and extruded under predetermined processing conditions.

| Ingredient | Composition | % Composition |
|---|---|---|
| Drug Substance | M.P. = 222° C., | 25 |
| | Log P = 3.48 | |
| Polyvinyl pyrrolidone | Solid hydrophilic | 62 |
| | polymer | |
| Polyethylene glycol | High boiling liquid | 5 |
| Caprylocaproyl | Emulsifier | 5 |
| polyoxyglycerides | | |
| α-Hydro-ω- | Anti-nucleating | 1 |
| hydroxypoly(oxyethylene)- | Agent | |
| poly(oxypropylene)- | | |
| poly-(oxyethylene)block | | |
| copolymer | | |
| Silicon dioxide | Anti-adherent | 2 |
| Total | | 100 |

The drug substance was successfully dissolved in the composition as was confirmed by the absence of crystallinity in the samples. When the sample was tested using a two-stage biorelevant dissolution method involving simulated fasted state and fed state dissolution media, more than 80% was released in 45 minutes under both conditions.

Dissolution

| Dissolution | Time | % Released | % Released |
|---|---|---|---|
| stages | (min) | Fasted state | Fed state |
| Stage 1 | 5 | 100 | 44 |
| (Stomach condition) | 10 | 102 | 51 |
| | 15 | 102 | 64 |
| Stage 2 | 20 | 69 | 72 |
| (Intestinal Condition) | 25 | 75 | 77 |
| | 30 | 84 | 77 |
| | 45 | 84 | 82 |
| | 60 | 87 | 86 |
| | 75 | 92 | 89 |
| | 90 | 93 | 78 |
| | 105 | 92 | 74 |

### Example 2

A composition containing a mixture of a drug substance, a solid hydrophilic polymer, a high boiling liquid, an emulsifier, an anti-nucleating agent and an anti-adherent was blended/granulated. The blend/granulation was then fed into a twin-screw extruder and extruded under predetermined processing conditions.

| Ingredient | Composition | % Composition |
|---|---|---|
| Drug Substance | M.P. 120° C., pKa = 11.5 | 21. |
| Polyvinyl pyrrolidone | Solid hydrophilic polymer | 60 |
| Polyethylene glycol | High boiling liquid | 6 |
| Caprylocaproyl | Emulsifier | 6 |
| polyoxyglyceride | | |
| Hydroxypropyl- | Anti-nucleating Agent | 4 |
| methylcellulose | | |
| Silicon dioxide | Anti-adherent | 3 |
| Total | | 100 |

The drug substance was successfully dissolved in the composition as was confirmed by the absence of crystallinity in the samples. When the sample was tested using simulated fasted state and fed state dissolution media, more than 95% was released in 90 minutes in both media.

Dissolution

| Time | % Released | % Released |
|---|---|---|
| (min) | Fasted state | Fed state |
| 15 | 26 | 20 |
| 30 | 56 | 45 |
| 60 | 91 | 83 |
| 90 | 100 | 95 |
| 120 | 100 | 96 |

### Example 3

A composition containing a mixture of the drug substance, a solid hydrophilic polymer, a high boiling liquid, an emulsifier, an anti-nucleating agent, an anti-adherent and an anti-oxidant was blended/granulated. Anti-oxidant was added to the base composition to impart stability on the drug substance without altering the functionality of the composition. The blend/granulation was then fed into a twin-screw extruder and extruded under predetermined processing conditions.

| Ingredient | Composition | % Composition |
|---|---|---|
| Drug Substance | pKa = 4.7, Log P = 6.9 | 20 |
| Polyvinyl pyrrolidone | Solid hydrophilic polymer | 47 |
| Polyethylene glycol | High boiling liquid | 6 |
| Polyoxy ethylene Sorbitan | Emulsifier | 5 |
| Monooleate | | |
| Cremophor RH 40 | Emulsifier | 12 |
| Hydroxypropyl- | Anti-nucleating Agent | 1 |
| methylcellulose | | |
| Silicon dioxide | Anti-adherent | 8 |
| Propyl gallate | Anti-oxidant | 1 |
| Total | | 100 |

The drug substance was successfully dissolved in the composition as was confirmed by the absence of crystallinity in the samples. When the sample was tested using a simulated gastric fluid dissolution method, more than 95% of the drug was released in 30 minutes.

Dissolution

| Time | % Released Simulated |
|---|---|
| (min) | Gastric fluid |
| 5 | 94 |
| 10 | 95 |
| 20 | 96 |
| 30 | 97 |
| 45 | 99 |
| 60 | 100 |

It has been discovered that the qualitative and quantitative composition of each component are critical to achieve solubilization of drug substances irrespective of their physicochemical properties. Removing one of the components or changing the ratio of each component to each other do not lead to the desired product. For example:
1) removal or reduction of the emulsifier amount led to precipitation;
2) removal or reduction of the solid hydrophilic polymer led to residual crystalline material in the product;
3) removal or reduction of the hydrophilic polymer led to formation of semi-solid material;
4) removal or reduction of antinucleating agent led to precipitation of the drug substance during dissolution; and
5) removal or reduction of anti-adherent led to processability and stability problems

Surprisingly, however, formulations consisting of all components at appropriate ratios as determined by the physicochemical properties of the drug substance provided solubility enhanced products irrespective of the physicochemical properties of the drug substances (i.e. melting point, log P, pKa), results which could not be achieved by any of the other technologies known in the art.

It is claimed:

1. A solid oral dosage form composition for enhancing bioavailability of poorly soluble drug substances, comprising: a drug substance, a solid hydrophilic polymer, a high boiling liquid, an emulsifier, an anti-nucleating agent, and an anti-adherent.

2. The composition in claim 1, wherein the weight ratio of the amount of the emulsifier relative to that of the solid hydrophilic polymer is between 1:50 and 50:1.

3. The composition in claim 1, wherein the weight ratio of the amount of the emulsifier relative to that of the high boiling liquid is between 1:20 and 20:1.

4. The composition in claim 1, wherein the weight ratio of the amount of the emulsifier relative to that of the anti-nucleating agent is between 1:50 and 50:1.

5. The composition in claim 1, wherein the weight ratio of the amount of the high boiling liquid relative to that of the anti-nucleating agent is between 1:20 and 20:1.

6. The composition in claim 1, wherein the weight ratio of the amount of the high boiling liquid relative to that of the solid hydrophilic polymer is between 1:50 and 50:1.

7. The composition in claim 1, wherein the weight ratio of the amount of the anti-nucleating agent relative to that of the solid hydrophilic polymer is between 1:1 and 1:200.

8. The composition in claim 1, wherein the amount of the anti-adherent relative to that of the total composition by weight is between 0.1% and 20%.

9. The composition of claim 1, wherein the amount of drug substance relative to that of the total composition is between 1% and 70%.

10. The composition of claim 1, wherein the drug substance that is BCS II or BCS IV according to Biopharmaceutical Classification System.

11. The composition in claim 1, wherein the drug substance is a member of the group consisting of acetazolamide, azathioprine, amphoterecin B, aprepitant, carbamazepine, carvedilol, cilostazol, cyclosporine, danzol, etavirine, famotidine, furosemide, hydrochlorthiazide, hydroxyzine, ibuprofen, indinavir, itraconazole, ketoconazole, ketoprofen, lopinavir, meloxicam, nelfinavir, piroxicam, ritonavir, and saquinavir, or a combination thereof.

12. The composition of claim 1, wherein the solid hydrophilic polymer is a member of the group consisting of a cellulose derivative that is hydroxypropyl cellulose, hydroxypropyl cellulose acetate succinate, or hydroxypropylmethyl cellulose; and a vinyl derivative that is polyvinyl pyrrolidone or polyvinyl pyrrolidone vinyl acetate, or a mixture thereof.

13. The composition of claim 1, wherein the high boiling liquid is a member of the group consisting of polyethylene glycol, propylene glycol, and diethylene glycol monoethyl ether, or a mixture thereof.

14. The composition of claim 1, wherein the emulsifier is a member of the group consisting of caprylocaproyl polyoxyglycerides, propylene glycol dicaprylocaprate, and polyoxyl castor oil or a mixture thereof.

15. The composition of claim 1, wherein the anti-nucleating agent is a member of the group consisting of low molecular weight hydrophilic polymer such as hydroxypropylmethyl cellulose and surfactants such as α-Hydro-ω-hydroxypoly(oxyethylene)-poly(oxypropylene).poly(oxyethylene) block copolymer, polyethylene glycol-20 sorbitan monolaurate, and polyethylene glycol-20 monostearate, or a mixture thereof.

16. The composition of claim 1, wherein the anti-adherent is a member of the group consisting of silicon dioxide, and talc, or a mixture thereof.

17. A process of preparing the composition of claim 1, comprising: using extrusion to mix the drug substance, the solid hydrophilic polymer, the high boiling liquid, the emulsifier, the anti-nucleating agent, and the anti-adherent.

18. The process of claim 17, wherein extrusion is a twin-screw high intensity mixing process that utilizes co-rotating or counter-rotating screw elements.
